(19) United States
(12) Patent Application Publication
(10) Pub. No.: US 2014/0273139 A1
(43) Pub. Date: Sep. 18, 2014

(54) Direct Fermentation of Biomass to Fuel Precursors

(71) Applicant: Innovative Metabolism Inc., Andover, MA (US)

(72) Inventor: Anthony A. Ferrante, Belmont, MA (US)

(73) Assignee: Innovative Metabolism Inc., Andover, MA (US)

(21) Appl. No.: 14/215,261

(22) Filed: Mar. 17, 2014

Related U.S. Application Data
(60) Provisional application No. 61/788,581, filed on Mar. 15, 2013.

(51) Int. Cl. C12P 7/10

(52) U.S. Cl. CPC C12P 7/10
USPC 435/165

(57) ABSTRACT

Featured herein are mixed solvent fermentation processes for efficiently generating sulfur-free fuels from low-cost raw materials.

This application claims the benefit of priority to U.S. Provisional Patent Application Ser. No. 61/788,581, filed Mar. 15, 2013; the contents of which are hereby incorporated by reference.

### BACKGROUND OF THE INVENTION

The biofuels market has grown substantially over the last decade. For example, the bioethanol market was estimated to be about 15,000,000 m³ in 2000 and about 70,000,000 m³ in 2009 with a value of $32 billion USD at $1.80/gal.

Further growth in the ethanol market, however is limited by the low net energy yield of ethanolic fermentation, high cost of production and low energy density compared with gasoline.

New processes for producing biofuel are needed.

### SUMMARY OF THE INVENTION

In one aspect is featured a mixed solvent fermentation process, which comprises the steps of: (a) culturing lignocellulosic biomass with a wood-rot fungus of the phylum Basidiomycota (for example a species within the genus Phanerochaete or Coprinopsis) in an appropriate bioreactor and under appropriate aerobic conditions for an appropriate amount of time to establish growth; and (b) converting the conditions within the bioreactor to anaerobic conditions to produce a vapor phase containing volatile solvents, such as ethanol, acetone and/or isopropanol. The process may be performed in a solid-state fermentation and at a temperature of at least about 37° C. The process may include the addition step of recovering volatile products from the vapor phase. The process may comprise the additional step of converting the conditions within the bioreactor back to aerobic conditions. In fact the conversion of culture conditions from aerobic to anaerobic may be performed repeatedly to optimize biomass degradation and fermentation.

In another aspect is featured a mixed solvent fermentation process, which comprises the steps of: (a) culturing lignocellulosic biomass with a wood-rot fungus of the phylum Basidiomycota in an appropriate bioreactor and under appropriate aerobic conditions for an appropriate amount of time to establish growth; and (b) introducing into the bioreactor an appropriate amount of a metabolic inhibitor to produce a vapor phase containing volatile solvents. The process may further comprise the step of removing the inhibitor and introducing sufficient oxygen into the bioreactor to establish aerobic conditions. In fact, the addition and removal of inhibitor and oxygen may be performed repeatedly to optimize biomass degradation and fermentation.

The processes described above may be carried out by wood-rot fungus of the phylum Basidiomycota alone or cultured in conjunction with other fungi, such as fungi of the Saccharomycetales order of Ascomycota including members of the Saccharomyces and Kluveromyces genera. For example, the different fungi can have differing substrate preferences enabling more complete biomass utilization.

Biomass may be pretreated prior to performance of the processes described herein, for example by a process such as steam explosion, hydrothermal pretreatment, or supercritical fluid expansion.

The processes described herein produce sulfur-free fuels that are efficiently generated from low-cost raw materials and are thus competitive with petroleum-derived diesel and jet fuel.

Other features and advantages will be apparent based on the following Detailed Description and Claims.

### BRIEF DESCRIPTION OF THE DRAWINGS

FIG. 1 is a process diagram for solid-state fermentation of lignocellulosic biomass by fungus. Biomass is expanded (100) to increase surface area; solid-state fermentation (105) is initiated; an optional cooling loop (110) recirculates air and re-injects condensate into the bioreactor for additional cooling; volatile solvents are periodically recovered (115) from the vapor phase; and product is purified and concentrated (120) using distillation.

FIG. 2 is a process diagram illustrating the recovery of non-volatile fermentation products from residual solids (200) including recovery and separation of water-soluble products (215) such as organic acids, lignin monomers, and residual sugars, and drying of non-water soluble material (205) for use as animal feed or compost (210).

FIG. 3a is a gas chromatography retention plot for a sample from an aerobic, submerged liquid culture of P. chrysosporium using glucose as the primary carbon source. It demonstrates that no ethanol was produced.

FIG. 3b is a gas chromatography retention plot for a sample from anaerobic, submerged liquid culture of P. chrysosporium using glucose as the primary carbon source. It demonstrates ethanol production.

FIG. 3c show a gas chromatography retention plot for a sample from aerobic, submerged liquid culture with nitric oxide (NO) bubbled through the medium of P. chrysosporium using glucose as the primary carbon source. It demonstrates ethanol production in the presence of oxygen.

FIG. 4 shows a gas chromatography retention plot for P. chrysosporium grown in liquid culture under oxygen-limited conditions and treated with rotenone. It demonstrates production of acetone, isopropanol and ethanol in the presence of a mitochondrial inhibitor.

FIG. 5 is a schematic representation of a solid-state fermentation apparatus that enables intermittent condensate collection and product recovery in which process gas is introduced through a filter (500) into a solid-state fermentation chamber (530), vapor is recirculated via valves (540) and (545) passing through sensors for oxygen (520) and carbon dioxide (515), through a pump (510), through a filter (505) and re-enters the chamber. Volatile products are collected either by changing the position of valves (540) and (545) so that they pass through a condenser (550). Alternatively, volatile products are collected from exhaust gas (535).

FIG. 6 is a graph showing total solvent production from a co-culture of P. chrysosporium and K. marxianus grown on cracked corn under solid-state conditions.

FIG. 7 is a graph showing the rate of solvent production (solid line) and the solvent yield (dashed line) for co-culture of C. cinerea and K. marxianus grown on corn stover under anaerobic conditions.

FIG. 8 is a graph showing the rate of solvent production per gram of biomass (solid line) and the solvent yield (dashed line) for a co-culture of P. chrysosporium and K. marxianus grown on corn stover under varying concentrations of carbon monoxide.

FIG. 9 is a graph showing the rate of solvent production per gram of biomass (solid line) and the solvent yield (dashed line) for P. chrysosporium grown on corn stover with carbon monoxide followed by recovery and subsequent anaerobic growth.

### DETAILED DESCRIPTION OF THE INVENTION

### General

Featured herein are processes for direct fermentation of lignocellulosic biomass to volatile products in a mixed solvent fermentation. Major components of the solvent product include ethanol, acetone, and isopropanol. The processes utilize wood-rot fungi in the phylum Basidiomycota that produce an array of cellulases and hemicellulases as well as lignin peroxidases, manganese peroxidases, copper radical oxidases, cellobiose dehydrogenase, and pyranose-2-oxidase that interact to promote rapid depolymerization of wood, grasses, and other lignocellulosic materials [See Martinez, D., et al., Genome, transcriptome, and secretome analysis of wood decay fungus Postia placenta supports unique mechanisms of lignocellulose conversion. Proc Natl Acad Sci U S A, 2009. 106(6): p. 1954-96; Vanden Wymelenberg, A., et al., Comparative transcriptome and secretome analysis of wood decay fungi Postia placenta and Phanerochaete chrysosporium. Appl Environ Microbiol, 2010. 76(11): p. 3599-610].

The processes are based on the ability of wood rot fungi to completely degrade lignocellulosic biomass under aerobic conditions [Muller, H. and W. Trosch, Screening of white-rot fungi for biological pretreatment of wheat straw for biogas production. Appl Microbiol Biotechnol, 1986. 24: p. 180-185; Reid, I.D., Biological Delignification of Aspen Wood by Solid-State Fermentation with the White-Rot Fungus Merulius tremellosus. Appl Environ Microbiol, 1985. 50(1): p. 133-9; Steenkjaer Hastrup, A., et al., The effect of CaCl₂ on growth rate, wood decay and oxalic acid accumulation in Serpula lacrymans and related brown-rot fungi. Holzforschung, 2006. 60: p. 339-345; Valaskova, V. and P. Baldrian, Degradation of cellulose and hemicelluloses by the brown rot fungus Piptoporus betulinus—production of extracellular enzymes and characterization of the major cellulases. Microbiology, 2006. 152(Pt 12): p. 3613-22; and Vanden Wymelenberg, A., et al., Comparative transcriptome and secretome analysis of wood decay fungi Postia placenta and Phanerochaete chrysosporium. Appl Environ Microbiol, 2010. 76(11): p. 3599-610] and ferment sugars to ethanol under anaerobic conditions [Kenealy, W. and D. Dietrich, Growth and fermentation responses of Phanerochaete chrysosporium to O₂ limitation. Enzyme and Microbial Technology, 2004. 34: p. 490-498; and Okamoto, K., et al., Production of ethanol by the white-rot basidiomycetes Peniophora cinerea and Trametes suaveolens. Biotechnol Lett, 2010. 32(7): p. 909-13].

Cellulases and hemicellulases depolymerize cellulose and hemicellulose, respectively, and their activity does not require oxygen. Lignin degradation is catalyzed by lignin proxidases and manganese peroxidases which are activated by hydrogen peroxide. Generation of hydrogen peroxide requires oxygen and is catalyzed by oxidases including copper radical oxidases and pyranose-2-oxidase [Kersten, P J. 1990. Glyoxal oxidase of Phanerochaete chrysosporium: Its characterization and activation by lignin peroxidase. Proc Nat. Acad. Sci. USA 81:2936-40.]

These novel processes are based on the discovery of inducible acetone-isopropanol fermentation in Phanerochaete chrysosporium and Coprinopsis cinerea. Both organisms are members of the class Agaricomycetes. P. chrysosporium is a member of the order Polyporales while C. cinerea is a member of the order Agaricales. Given the level of genetic separation between these organisms, it is expected that the ability to ferment lignocellulosic biomass and other carbon sources to acetone and isopropanol is widely distributed among the members of the phylum Basidiomycota. This is the first identification of significant isopropanol production during fermentation outside of the genus Clostridium [Survase, S. A., et al., Continuous production of isopropanol and butanol using Clostridium beijerinckii DSM 6423. Appl Microbiol Biotechnol. 91(5): p. 1305-13.] and the first identification of this ability in a facultative anaerobe.

These novel processes are also based on the development of methods that enable fermentative solvent production while providing sufficient oxygen for lignin degradation. According to one method, Basidiomycota is grown under anaerobic conditions. In this method fungal growth is first established under aerobic conditions. Establishment of growth can be measured by monitoring the rate of carbon dioxide production. Under aerobic conditions 6 carbon dioxide molecules are produced for every glucose equivalent that is respired. Once growth is established air is no longer fed into the bioreactor, oxygen is depleted, and the conditions become anaerobic. Production of volatile solvents can be monitored periodically.

Productivity, i.e. the rate of solvent production per gram of biomass per unit time, declines over time as easily accessible carbohydrates are consumed. Fungal metabolic activity may also decline during extended periods under anaerobic conditions. Activity can be restored by periodically introducing air into the bioreactor. As aerobic conditions are re-established the fungi become more metabolically active and are able to use the available oxygen to degrade lignin, thus exposing more fermentable carbohydrate. It is likely that production of cellulose and hemicellulose degrading enzymes is enhanced under aerobic conditions. Once growth is re-established the bioreactor is again switched to anaerobic conditions and production of volatile solvents resumes.

It should be apparent to persons skilled in the art that the composition of the fermentation substrate can be amended using a variety of additives that may enhance fungal growth, increase solvent production rate, increase solvent yield, and/or alter the balance of solvent products. Additives may include, but are not limited to, salts and trace elements such as sodium citrate, potassium nitrate, ammonium phosphate, magnesium sulfate, calcium chloride, citric acid, zinc sulfate, Ammonium iron(II) sulfate, copper sulfate, manganese sulfate, boric acid, and sodium molybdate. Fungal growth can be enhanced by addition of sources of protein including yeast extract, or food waste. Basidiomycete growth and fermentation may also be enhanced by addition of lignin and other byproducts of wood pulping, or tree bark.

Other processes rely on the use of a metabolic inhibitor such as carbon monoxide to inhibit respiration thus enabling fermentation in the presence of oxygen. Cytochrome c oxidase is a key regulator of respiration in mitochondria. Growth of facultative anaerobes such as Basidiomycota in the presence of an appropriate inhibitor can redirect cellular energetics toward fermentative growth in the presence of oxygen, thus enabling fungal fermentation under conditions that provide sufficient oxygen for lignin degradation.

For example, initial fungal growth may be established under aerobic conditions. Once growth is established an inhibitor such as carbon monoxide is introduced into the system. Productivity declines over time as easily accessible carbohydrates are consumed. Fungal metabolic activity may also decline during extended periods in the presence of metabolic inhibitors. Activity may be restored by removing the inhibitor and introducing sufficient oxygen to establish aerobic growth. Once growth is re-established, inhibitor may be again added to the bioreactor and production of volatile solvents resumes.

In the event that complete inhibition of fungal respiration reduces its wood degradation activity it should be possible to titrate the mitochondrial inhibitor to reduce but not eliminate respiration. Glycolysis would generate both pyruvate and reducing equivalents (NADH+H⁺) under those conditions faster than they are consumed. Excess pyruvate would be shunted toward ethanol production in a process that recycles NADH+H⁺ to NAD.

A consortium of yeasts and fungi may be used to increase the rate and yield (i.e. the percentage of biomass converted to solvent product) of lignocellulosic biomass fermentation. For example, consortia may include more than one member of the phylum Basidiomycota whose members have differing substrate preferences. One exemplary consortium includes P. chrysosporium and C. cinerea. While C. cinerea has similar enzymatic capabilities to P. chrysosporium, it is generally a late-succession degradative organism preferentially attacking partially degraded material. Inclusion of a yeast of the phylum Ascomycota in fungal consortia results in higher levels of fungal activity, measured by carbon dioxide production, as well as higher productivity and yield. This increase in activity may be due to both autolysis of the yeast which provides additional nutrients to the Basidiomycota, and due to the yeast scavenging sugars released from the biomass by the Basidiomycota and fermenting those sugars to ethanol.

The processes may be performed under solid-state fermentation conditions, in which the biomass is kept humid but is not immersed in liquid. Conditions in a solid-state fermentation bioreactor mimic the natural habitat of wood-rot Basidiomycota more closely than the submerged liquid fermentation which is standard in the fuel ethanol fermentation industry. Use of solid-state conditions has several key advantages for the production of mixed solvent products from lignocellulosic materials by Basidiomycota. These include reduced water use; reduced energy requirements for mixing and thermal control as compared with submerged liquid systems; superior gas exchange between fungal mycelia and the process gas mixture, the ability to monitor fermentation by measurement of carbon dioxide production and by the concentration of volatile fermentation products in the vapor phase; and the ability to collect volatile solvent products from the vapor phase with high initial concentration and high purity.

Thermotolerant fungal and yeast species may be used in the disclosed processes and bioreactor temperatures maintained above 37° C. Most preferably bioreactor temperatures are maintained at temperatures ranging between about 37° C. and 48° C. Few members of the white rot fungi in the phylum Basidiomycota are known to tolerate elevated temperatures. P. chrysosporium was chosen initially for its ability to grow at temperatures up to 48° C. A strain of C. cinerea was isolated that exhibits similar rates of growth at 37° C. and 44° C. K. marxianus strains are known to grow on a wide range of sugars and at temperatures of up to 49° C. or higher [Nonklang, S., et al., High-temperature ethanol fermentation and transformation with linear DNA in the thermotolerant yeast Kluyveromyces marxianus DMKU3-1042. Appl Environ Microbiol, 2008. 74(24): p. 7514-21]. Advantages of using higher temperature fermentation include faster biomass decomposition and more favorable partitioning of volatile solvent products to the vapor phase for recovery at high concentration and high purity.

Lignocellulosic biomass with large particle size may be used in the disclosed processes. Current fermentation processes use energy to reduce biomass to fine particulate, typically 1 mm particle size or smaller in order to increase accessibility for enzymatic hydrolysis. The use of larger particle sizes in the disclosed processes reduces energy and cost associated with milling. It is possible, however, to increase productivity during fermentation by consortia of Basidiomycota and Ascomycota by increasing the exposed surface area of the cellulose and hemicellulose. This may be accomplished using techniques such as, but not limited to, steam explosion or supercritical fluid explosion. These processes penetrate the cellulose rich fibers in lignocellulosic biomass. Upon release of pressure, rapid expansion of the pressurized water vapor or supercritical fluid disrupts cellulosic fibers thus significantly increasing surface area.

While the disclosed processes focus on the production of volatile solvents including ethanol, acetone, and isopropanol from lignocellulosic materials using solid-state fermentation, the use of members of the phylum Basiodiomycota, alone or in consortia with members of the phylum Ascomycota, may also produce acetone and isopropanol from starch and simple sugars either in solid-state fermentation or in submerged liquid fermentation.

In addition, members of the phylum Basidiomycota are known to produce a wide array of products. While the subject invention focuses on production of volatile solvents it does not preclude the harvesting of other commercially valuable products. Such products may include but are not limited to: acetic acid; oxalic acid; malic acid; lignin degradation products; ethyl acetate. In addition, residual solids may be collected for use as animal feed or as compost.

### Exemplary Embodiments

In certain embodiments, the invention relates to a mixed solvent fermentation process comprising the steps of: (a) culturing lignocellulosic biomass with wood-rot fungi of the phylum Basidiomycota in an appropriate bioreactor and under appropriate aerobic conditions for an appropriate amount of time to establish growth; and (b) converting the bioreactor to anaerobic conditions to produce a vapor phase containing volatile solvents.

In certain embodiments, the invention relates to any one of the aforementioned processes, wherein the vapor phase contains ethanol, acetone and/or isopropanol.

In certain embodiments, the invention relates to any one of the aforementioned processes, wherein the wood-rot fungi includes a species within the genus Phanerochaete.

In certain embodiments, the invention relates to any one of the aforementioned processes, wherein the wood-rot fungi includes a species within the genus Coprinopsis.

In certain embodiments, the invention relates to any one of the aforementioned processes, which is a solid-state fermentation.

In certain embodiments, the invention relates to any one of the aforementioned processes, wherein the fermentation is performed at a temperature of at least about 37° C.

In certain embodiments, the invention relates to any one of the aforementioned processes, which further comprises the step of recovering volatile products from the vapor phase.

In certain embodiments, the invention relates to any one of the aforementioned processes, further comprising the step of converting the bioreactor back to aerobic conditions.

In certain embodiments, the invention relates to any one of the aforementioned processes, wherein the process is performed repeatedly.

In certain embodiments, the invention relates to a mixed solvent fermentation process comprising the steps of: (a) culturing lignocellulosic biomass with wood-rot fungi of the phylum Basidiomycota in an appropriate bioreactor and under appropriate aerobic conditions for an appropriate amount of time to establish growth; and (b) introducing into the bioreactor an appropriate amount of a metabolic inhibitor to produce a vapor phase containing volatile solvents.

In certain embodiments, the invention relates to any one of the aforementioned processes, wherein the vapor phase contains ethanol, acetone and/or isopropanol.

In certain embodiments, the invention relates to any one of the aforementioned processes, wherein the wood-rot fungus includes a species within the genus Phanerochaete.

In certain embodiments, the invention relates to any one of the aforementioned processes, wherein the wood-rot fungi includes a species within the genus Coprinopsis.

In certain embodiments, the invention relates to any one of the aforementioned processes, which is a solid-state fermentation.

In certain embodiments, the invention relates to any one of the aforementioned processes, wherein the fermentation is performed at a temperature of at least about 37° C.

In certain embodiments, the invention relates to any one of the aforementioned processes, further comprising the step of recovering volatile products from the vapor phase.

In certain embodiments, the invention relates to any one of the aforementioned processes, further comprising the step of removing the inhibitor and introducing sufficient oxygen into the bioreactor to establish aerobic conditions.

In certain embodiments, the invention relates to any one of the aforementioned processes, which is performed repeatedly.

In certain embodiments, the invention relates to any one of the aforementioned processes, wherein the mitochondrial inhibitor is a gas.

In certain embodiments, the invention relates to any one of the aforementioned processes, wherein the mitochondrial inhibitor is selected from the group consisting of carbon monoxide, nitric oxide, hydrogen sulfide and hydrogen cyanide, alone or in combination.

In certain embodiments, the invention relates to any one of the aforementioned processes, wherein the wood-rot fungus of the phylum Basidiomycota is cultured in conjunction with at least one fungus that is a member of the Saccharomycetales order of Ascomycota.

In certain embodiments, the invention relates to any one of the aforementioned processes, wherein the Saccharomycetales fungi are of the genus Saccharomyces and Kluveromyces.

In certain embodiments, the invention relates to any one of the aforementioned processes, wherein the members of the wood-rot fungus of the phylum Basidiomycota and the fungus that is a member of the Saccharomycetales order of Ascomycota have differing substrate preferences enabling more complete biomass utilization.

In certain embodiments, the invention relates to any one of the aforementioned processes, wherein the wood-rot fungus of the phylum Basidiomycota is Phanerochaete chrysosporium or Coprinopsis cinerea.

In certain embodiments, the invention relates to any one of the aforementioned processes, wherein the fungus is Kluyveromyces marxianus.

In certain embodiments, the invention relates to any one of the aforementioned processes, wherein fermentation rates are enhanced by pretreating the lignocellulosic biomass.

In certain embodiments, the invention relates to any one of the aforementioned processes, wherein the pretreatment is a member selected from the group consisting of: steam explosion, hydrothermal pretreatment, or supercritical fluid expansion.

### Definitions

As used herein, the following words and phrases have the meanings provided below.

“Ascomycota fungi refer to a phylum of higher fungi, which along with Basidomycota make up the Dikarya fungi subkingdom. There are a variety of orders within the Ascomycota fungi, including Saccharomycetales, which includes the Saccharomyces and Kluveromyces genera (e.g. K. marxianus).

The term “bioreactor” refers to any vessel or configuration in which lignocellulosic biomass is maintained in a humid environment but not submerged in liquid. Specific designs may include batch processors, continuous-feed processors, the use of mixers to reduce biomass clumping, and other configurations known to those familiar with the art [Mitchell, D., N. Krieger, and M. Berovic, eds. Solid-State Fermentation Bioreactors: Fundamentals of Design and Operation. 1 edition ed. 2006, Springer: Berlin Heidelberg. 485].

“Electron transport chain” refers to a series of protein complexes that accept electrons from NADH+H⁺ and FADH₂ and uses that energy to establish and maintain a proton (chemiosmotic) gradient. Energy stored in the proton gradient is used for energy generation and for other purposes.

“Facultative anaerobe” refers to an organism or cell that is capable of either aerobic or anaerobic growth or cell maintenance.

“Fermentation” refers to a metabolic pathway that uses organic molecules as terminal electron acceptors resulting in formation of alcohols and/or acids. Fermentation is a form of anaerobic metabolism in which sugars such as glucose are oxidized to pyruvate. Organic molecules are used as the final electron acceptor during fermentation enabling re-oxidation of NADH+H⁺ generated during glycolysis to NAD⁺. In the case of ethanol fermentation pyruvate is decarboxylated to yield acetaldehyde which is reduced by alcohol dehydrogenase to form ethanol. Aerobic respiration generates significantly more energy per glucose molecule. Facultative anaerobes such as yeasts, which can grow either aerobically or anaerobically, will therefore preferentially respire when oxygen is present.

“Glycolysis” refers to the first stage in sugar metabolism used by most organisms in which glucose is converted biochemically to pyruvate in a process that generates ATP and NADH+H⁺.

“Lignocellulosic biomass” refers to any material that is a natural product or is derived from a natural product and contains cellulose and lignin. It may contain but is not limited to wood, wood-pulp, forestry products, forestry or agricultural waste, grass, other grassy plants, leaf litter, municipal compost, municipal waste, food waste and waste paper.

“Mitochondrial inhibitor” refers to a compound that inhibits respiration in a eukaryotic organism via interaction with components of the electron transport system or the tricarboxylic acid cycle. Inhibition may be competitive or irreversible. The concentration of a reversible inhibitor may be modulated to allow sufficient respiration for cellular growth and additional NADH+H⁺ generation while still enabling the redirection of the majority of carbon flux through fermentation, which may result in higher rates of fermentation than incubation under strictly fermentative conditions.

A mitochondrial inhibitor may, for example, interact with a cytochrome c oxidase (also known as complex IV). This complex is the site at which oxygen binds and is reduced to water. This is one of the key regulatory components of electron transport. Inhibitors of cytochrome c oxidase include nitric oxide, hydrogen sulfide, cyanide, azide, carbon monoxide, and formic acid. Alternatively, a mitochondrial inhibitor may interact with cytochrome c reductase (also known as complex III, cytochrome bc₁ complex, or coenzyme Q:cytochrome c—oxidoreductase), another key component in the electron transport chain. Transport of electrons from this complex to cytochrome c oxidase results in translocation of 4 protons. Compounds that specifically inhibit the cytochrome c reductase complex, include antimycin A, myxothiazol, and stigmatellin. It will be apparent to those schooled in the art that inhibitors of other steps in respiration may also be used to redirect carbon and energy flux toward fermentation. These include but are not limited to malonate, a competitive inhibitor of succinate dehydrogenase in the citric acid cycle, and rotenone, a metabolic poison.

“Mixed-solvent fermentation” refers to biological production of a combination of at least two members of the group ethanol, acetone, and isopropanol.

“Respiration” refers to growth or cell maintenance using the electron transport chain.

“Solid-state fermentation” refers to a process in which the material to be fermented is maintained under moist or humid conditions but is not substantially immersed in aqueous media.

“The tricarboxylic acid cycle” refers to a series of biochemical steps that begin with a condensation reaction to generate citrate from acetyl-CoA and oxaloacetate, and that results in the release of 2 molecules of CO₂ and reduction of 2 molecules of NAD⁺ to NADH+H⁺, one molecule of NADP⁺ to NADH+H⁺, and one molecule of FAD to FADH₂. This is the major pathway for energy generation from carbohydrates, fats and proteins in aerobic organisms.

“Wood rot fungi” refer to fungi that are capable of degrading wood. Certain wood rot fungi are members of the phylum Basidomycota. The two major classes of wood rot basidiomycetes are differentiated by their ability to degrade lignin. White rot fungi, such as members of the Phanerochaete genus (e.g. P. chrysosporium) and Coprinopsis genus (e.g. C. cinerea), are capable of using both the cellulose and lignin components of wood although they are incapable of growth on lignin as a sole source of carbon [Reid, I. D., Biological Delignification of Aspen Wood by Solid-State Fermentation with the White-Rot Fungus Merulius tremellosus. Appl Environ Microbiol, 1985. 50(1): p. 133-9; and Vanden Wymelenberg, A., et al., Comparative transcriptome and secretome analysis of wood decay fungi Postia placenta and Phanerochaete chrysosporium. Appl Environ Microbiol, 2010. 76(11): p. 3599-610]. Brown rot fungi efficiently hydrolyze cellulose and hemicellulose from untreated wood and extensively modify but do not significantly remove lignin [Martinez, D., et al., Genome, transcriptome, and secretome analysis of wood decay fungus Postia placenta supports unique mechanisms of lignocellulose conversion. Proc Natl Acad Sci U S A, 2009. 106(6): p. 1954-9; Valaskova, V. and P. Baldrian, Degradation of cellulose and hemicelluloses by the brown rot fungus Piptoporus betulinus—production of extracellular enzymes and characterization of the major cellulases. Microbiology, 2006. 152(Pt 12): p. 3613-22; Vanden Wymelenberg, A., et al., Comparative transcriptome and secretome analysis of wood decay fungi Postia placenta and Phanerochaete chrysosporium. Appl Environ Microbiol, 2010. 76(11): p. 3599-610; Illman, B., ed. Oxidative degradation of wood by brown-rot fungi. Active Oxygen/Oxidative Stress and Plant Metabolism, ed. E. Pell and K. Steffen. 1991, American Society of Plant Physiologists; and Wang, W., J. Liu, and P. Gao, A Peptide-mediated Fenton reaction in wood-degrading fungi. Chinese Chemical Letters, 2002. 13(8): p. 773-776]. Both classes of basidiomycetes generate free radicals, including hydroxyl radicals, to aid in rapid depolymerization of cellulose well before the porosity of the wood is sufficient to permit diffusion of cellulase enzymes [Vanden Wymelenberg, A., et al., Comparative transcriptome and secretome analysis of wood decay fungi Postia placenta and Phanerochaete chrysosporium. Appl Environ Microbiol, 2010. 76(11): p. 3599-610;].

White rot basidiomycetes produce a broad array of cellulases and hemicellulases as well as lignin peroxidases, manganese peroxidases, copper radical oxidases, cellobiose dehydrogenase, and pyranose-2-oxidase that interact to promote rapid depolymerization of wood [Martinez, D., et al., Genome, transcriptome, and secretome analysis of wood decay fungus Postia placenta supports unique mechanisms of lignocellulose conversion. Proc Natl Acad Sci U S A, 2009. 106(6): p. 1954-9; and Vanden Wymelenberg, A., et al., Comparative transcriptome and secretome analysis of wood decay fungi Postia placenta and Phanerochaete chrysosporium. Appl Environ Microbiol, 2010. 76(11): p. 3599-610]. Brown rot fungi appear to have repeatedly evolved from white rot fungi. They produce an array of hemicellulases but are deficient in cellulases [Vanden Wymelenberg, A., et al., Comparative transcriptome and secretome analysis of wood decay fungi Postia placenta and Phanerochaete chrysosporium. Appl Environ Microbiol, 2010. 76(11): p. 3599-610]. The brown rot fungi appear to rely more heavily on oxidative mechanisms for cellulose depolymerization. A peptide-mediated Fenton reaction in which extracellular Fe(II) and H₂O₂ produced by the fungus react to generate hydroxyl radicals has been proposed [Wang, W., J. Liu, and P. Gao, A Peptide-mediated Fenton reaction in wood-degrading fungi. Chinese Chemical Letters, 2002. 13(8): p. 773-776].

“Wood rot fungi” include not only wild-type isolates, but also those engineered, for example to ferment more efficiently; for example by over-expression of alcohol dehydrogenase, or by overexpression of sugar transporters. Growth of a genetically engineered variant in a low oxygen environment and in the presence of a mitochondrial inhibitor may yield rapid degradation of woody biomass with simultaneous ethanol production.

All publications mentioned herein are hereby incorporated by reference in their entirety as if each individual publication was specifically and individually indicated to be incorporated by reference. In case of conflict, the present application, including any definitions herein, will control.

The invention, now being generally described, will be more readily understood by reference to the following examples, which are included merely for purposes of illustration of certain aspects and embodiments of the present invention, and are not intended to limit the invention.

### EXAMPLES

### Example 1

### Submerged Liquid Fermentation in the Presence of Air and Gaseous Mitochondrial Inhibitor

In this experiment P. chrysosporium was grown from conidiospores in submerged liquid culture. The growth medium was Highley's Basal salts [Wymelenberg, A. V., et al., The Phanerochaete chrysosporium secretome: database predictions and initial mass spectrometry peptide identifications in cellulose-grown medium. J Biotechnol, 2005. 118(1): p. 17-34.] supplemented with 0.1% yeast extract (YE) and 2% glucose. Conidiospores were inoculated in liquid medium in 3 neck flasks. The temperature was maintained at 37° C. The cultures were mixed using magnetic stir bars and gasses (air, nitrogen, and air supplemented with nitric oxide (NO)) were bubbled through the medium using stainless steel needles. Fungi were grown: 1) aerobically; 2) anaerobically; and 3) aerobically with NO delivered to a target concentration of 1 μM. Cultures were sampled daily and analyzed using GC-MS (FIG. 3). P. chrysosporium grew rapidly forming large, encapsulated masses of material. No ethanol was observed in the aerobic culture (FIG. 3a). Ethanol peaks were observed for anaerobic (FIG. 3b) and NO-treated (FIG. 3c) fungal cultures.

The identity of the ethanol peak was confirmed by MS analysis. Samples were transferred to glass septum vials and heated to 60° C. A gas-tight syringe was used to sample 50 μl from the headspace. Samples were analyzed on a 6890N GC system with a 5975 Mass Selective Detector (Agilent) using a PH Innowax column. Nitric oxide appeared to enable ethanol production in the presence of dissolved oxygen.

### Example 2

### Submerged Liquid Fermentation in the Presence of Air and the Mitochondrial Inhibitor Rotenone

In this experiment P. chrysosporium was grown from conidiospores in submerged liquid culture. The growth medium was Highley's Basal salts [Wymelenberg, A. V., et al., The Phanerochaete chrysosporium secretome: database predictions and initial mass spectrometry peptide identifications in cellulose-grown medium. J Biotechnol, 2005. 118(1): p. 17-34.] supplemented with 0.1% yeast extract (YE) and 2% glucose. Conidiospores were inoculated in 50 ml of liquid medium in a 250 ml Erlenmyer flask which was then capped with a 3-piece fermentation lock to prevent evaporation of volatile products and to allow the culture to incubate in a reduced oxygen environment. The culture was incubated statically.

A large mass of fungus was produced and remained suspended near the top of the medium. Samples were collected, transferred to glass septum vials and heated to 60° C. Samples were analyzed on a 6890N GC system with a 5975 Mass Selective Detector (Agilent) using a PH Innowax column. Three peaks were observed and were found to correspond to acetone, isopropanol, and ethanol (FIG. 4). Standard curves were performed for each solvent. The concentrations in the fermentation broth were determined to be 0.74 mM for acetone, 1.8 mM for isopropanol, and 1.9 mM for ethanol. This experiment provided the first indication that P. chrysosporium was capable of fermenting sugars to acetone and isopropanol.

### Example 3

### Construction of a Solid-State Fermentation System

Formation of large, encapsulated fungal masses in submerged liquid culture caused several problems related to the difficulty of delivering inhibitors to cells located on the interior of the fungal structures. A solid-state system was developed (FIG. 5) based on the rotating drum system described by Kalogeris et al. [Kalogeris, E., et al., Design of a solid-state bioreactor for thermophilic microorganisms. Bioresource Technology, 1999. 67: p. 313-315].

The chamber was constructed using a 6″ stainless steel pipe tee. Biomass is contained in an inner rotating drum formed from stainless steel mesh with a 1 mm screen size. An electric motor rotates the drum for 1 minute once every 3 hours. That implementation ample time is allowed for: mycelial infiltration of particulate biomass; and for adequate mixing to prevent clumping that would inhibit gas exchange.

Other features of the system included an air recirculation system driven by a diaphragm pump, and oxygen and carbon dioxide sensors to enable estimation of metabolic activity. Air, nitrogen, and gaseous inhibitors were delivered via Teflon tubing and were metered using MKS mass flow controllers and meters. Exhaust gas was passed through a chilled condenser to harvest volatile solvent products, mainly ethanol, acetone, and isopropanol.

The air recirculation system enables sensitive measurement of CO₂ and O₂, and can also be used to efficiently recover solvent products by condensation. Calculations aimed at establishing a theoretical maximum solvent product concentration were performed that assumed near saturation for both water and ethanol in the head space. At 42° C. the headspace gasses contain 0.057 g water/L and 0.192 g ethanol/L based on their vapor pressures. At saturation condensate collected from a 0° C. condenser would therefore contain 0.053 g water and 0.17 g ethanol from each liter of processed headspace gas for a final ethanol concentration of 76%. Early experiments reached a peak concentration of 17% solvent in exhaust condensates.

It is clear that many variations on this design would be effective. Addition of sensors capable of measuring vapor-phase solvent concentration in real time in combination with automated valves and process monitoring algorithms could enable process automation.

### Example 4

### P. Chrysosporium Grown on Cracked Corn Using Solid-State Fermentation

Cracked corn was used for initial experiments. Cracked corn is high in starch and is readily fermented by a wide range of microorganisms. Vogels salts [Gold, M. and T. Cheng, Conditions for fruit body formation in the white rot basidiomycete Phaerochaete chrysosporium. Arch. Microbiol., 1979. 121: p. 37-41.] was added to 200 g cracked corn in a ratio of 3 ml salts per gram of cracked corn. The mixture was autoclaved for 1 hour. After cooling a mixture of P. chrysosporium conidiospores and mycelia harvested from agar medium was added and the inoculated cracked corn was sterilely transferred to the rotating drum of the solid-state bioreactor. The bioreactor was sealed and placed in a 42° C. incubator.

Air was flowed through the system for the first day of the fermentation. The air was shut off and the system was allowed to become anaerobic on Day 2. Nitrogen was flowed through the system to maintain anaerobic conditions and to enable collection of solvents from the exhaust gas. Fermentation was continued for 28 days. Ethanol, acetone, and isopropanol production was measured using an HPLC system with a Bio-Rad Aminex HPX-87C carbohydrate analysis column. Ethanol comprised 75% of the final product. Acetone and isopropanol comprised 25% of the solvent product.

### Example 5

### Consortium of P. Chrysosporium and K. Marxianus Grown on Cracked Corn Using Solid-State Fermentation

Solid-state fermentation was set up as described in Example 4 except that 1 ml of K. marxianus from an overnight culture in YPD broth was also added to the cracked corn. After growth was established under aerobic conditions fermentation was initiated using low-oxygen conditions. Productivity during the first 26 days of fermentation reached a peak productivity of 4.5×10⁻⁵ g solvent/hour/g biomass remaining and peak yield of approximately 14% (FIG. 6). The solvent ratio was 64% ethanol, 21% acetone, and 15% isopropanol.

### Example 6

### Consortium of P. Chrysosporium and K. marxianus Grown on Cellulose Blotter Paper Using Solid-State Fermentation

Cellulose blotter paper with a 3 mm thickness was cut into approximately 1 cm squares. 200 ml of Vogel's salts [Gold, M. and T. Cheng, Conditions for fruit body formation in the white rot basidiomycete Phaerochaete chrysosporium. Arch. Microbiol., 1979. 121: p. 37-41.] was added to 200 g of paper and the paper was autoclaved for 30 min. After cooling a mixture of P. chrysosporium conidiospores and mycelia harvested from agar medium, and 1 ml of K. marxianus from an overnight culture in YPD broth were added to the paper. The paper was sterilely transferred to the rotating drum of the solid-state bioreactor. The bioreactor was sealed and placed in a 42° C. incubator.

After growth was established under aerobic conditions the input gas mix was changed to deliver 0.28 nmol of oxygen/hr. A total of 14 g of filter paper was consumed in 28 days and 0.33 g of solvent was produced. The solvent ratio was 80% ethanol, 11% acetone, and 9% isopropanol.

### Example 7

### Consortium of C. Cinerea and K. Marxianus Grown on Corn Stover

Corn stover was chopped into approximately 1 cm segments and further chopped using a food processor. A volume of Vogel's salts [Gold, M. and T. Cheng, Conditions for fruit body formation in the white rot basidiomycete Phaerochaete chrysosporium. Arch. Microbiol., 1979. 121: p. 37-41.] equal to the mass of the stover was added and the mixture was autoclaved for 1 hour. After cooling a mixture of P. chrysosporium conidiospores and mycelia harvested from agar medium, and a mixture of C. cinerea mycelia and basidiospores was added to the stover. The mixture was transferred to the rotating drum of the solid-state bioreactor under sterile conditions. The bioreactor was sealed and placed in a 44° C. incubator.

During the first 16.6 days productivity averaged 4.8×10⁻⁴ ml/day/g starting material and yield was 3.9% of biomass consumed (FIG. 7). The solvent ratio was 75% ethanol, 19% acetone, and 6% isopropanol.

### Example 8

### Consortium of P. Chrysosporium and K. Marxianus Grown on Corn Stover in the Presence of Carbon Monoxide (CO)

In this example, solvent-producing fermentation was initiated by introduction of a mix of air and CO, a competitive inhibitor of cytochrome c oxidase. Corn stover was prepared as described in Example 7. A mixture of P. chrysosporium conidiospores and mycelia harvested from agar medium, and 1 ml of K. marxianus from an overnight culture in YPD broth were added to the stover. The mixture was transferred to the rotating drum of the solid-state bioreactor under sterile conditions. The bioreactor was sealed and placed in a 44° C. incubator.

Growth was established under aerobic conditions (10 ml/min of air was flowed through the bioreactor for 17 hours). Fermentation was initiated by flowing a mixture comprised of 90% CO and 10% air through the bioreactor. During the first 27 days productivity peaked at 2.3×10⁻⁵ g solvent/hr/g biomass remaining and yield for the period was 5.7% of biomass consumed (FIG. 8). Ethanol was the only solvent produced in significant quantities during that period.

### Example 9

### P. Chrysosporium Grown on Corn Stover in the Presence of Carbon Monoxide (CO)

In this example solvent fermentation was initiated by introduction of a mix of 10% air and 90% CO. Corn stover was prepared as described in Example 7. P. chrysosporium conidiospores and mycelia harvested from agar medium were added to the stover and the mixture was transferred to the rotating drum of the solid-state bioreactor under sterile conditions. The bioreactor was sealed and placed in a 44° C. incubator. The volume of gas flow remained constant at 10 ml/min for the entire experiment.

The fermentor was run aerobically for 45 hours to establish growth. Between hour 45 and Day 44 a gas mix comprised of 10% air and 90% CO was flowed through the system. Fungal metabolic activity was restored by flowing air through the system from Day 51-55. From Day 57-65 the fermentation was run anaerobically in the absence of CO. Fungal metabolic activity was again restored by aerobic growth from Day 65-68, and then anaerobic fermentation was continued from Day 68-82. Productivity and yield were approximately 10-fold lower than achieved in Example 8 demonstrating the importance of co-culture of K. marxianus with the Basidiomycota (FIG. 9). Each period of aerobic growth showed little or no solvent production. Restoration of fermentation after aerobic growth resulted in restored productivity and yield.

In previous examples productivity declined over a period of 2-4 weeks. This experiment demonstrates that productivity can be restored after its decline by re-establishment of aerobic conditions until metabolic activity recovers. Fermentation of lignocellulosic biomass can be restored by establishing anaerobic conditions. It is notable that the solvent mix during fermentation in the presence of CO (through Day 44) was 72% ethanol and 28% acetone with only trace amounts of isopropanol. When fermentation was re-established using anaerobic conditions on Day 57 the solvent ratios changed: 15% ethanol, 6% acetone, and 79% isopropanol. Given the higher value of acetone and isopropanol, the ability to alter the product mix by altering the fermentation conditions may be a powerful tool.

1. A mixed solvent fermentation process comprising the steps of: (a) culturing lignocellulosic biomass with wood-rot fungi of the phylum Basidiomycota in an appropriate bioreactor and under appropriate aerobic conditions for an appropriate amount of time to establish growth; and (b) converting the bioreactor to anaerobic conditions to produce a vapor phase containing volatile solvents.

2. The process of claim 1, wherein the wood-rot fungi includes a species within the genus Phanerochaete.

3. The process of claim 1, wherein the wood-rot fungi includes a species within the genus Coprinopsis.

4. The process of claim 1, which is a solid-state fermentation.

5. The process of claim 1, wherein the fermentation is performed at a temperature of at least about 37° C.

6. The process of claim 1, which further comprises the step of recovering volatile products from the vapor phase.

7. The process of claim 1, further comprising the step of converting the bioreactor back to aerobic conditions.

8. The process of claim 7, wherein the process is performed repeatedly.

9. A mixed solvent fermentation process comprising the steps of: (a) culturing lignocellulosic biomass with wood-rot fungi of the phylum Basidiomycota in an appropriate bioreactor and under appropriate aerobic conditions for an appropriate amount of time to establish growth; and (b) introducing into the bioreactor an appropriate amount of a metabolic inhibitor to produce a vapor phase containing volatile solvents.

10. The process of claim 9, wherein the wood-rot fungus includes a species within the genus Phanerochaete.

11. The process of claim 9, which is a solid-state fermentation.

12. The process of claim 9, wherein the fermentation is performed at a temperature of at least about 37° C.

13. The process of claim 9, which further comprises the step of recovering volatile products from the vapor phase.

14. The process of claim 9, further comprising the step of removing the inhibitor and introducing sufficient oxygen into the bioreactor to establish aerobic conditions.

15. The process of claim 14, which is performed repeatedly.

16. The process of claim 9, wherein the mitochondrial inhibitor is a gas.

17. The process of claims 1, wherein the wood-rot fungus of the phylum Basidiomycota is cultured in conjunction with at least one fungus that is a member of the Saccharomycetales order of Ascomycota.

18. The process of claim 17, wherein the Saccharomycetales fungi are of the genus Saccharomyces and Kluveromyces.

19. The process of claim 17, wherein the members of the wood-rot fungus of the phylum Basidiomycota and the fungus that is a member of the Saccharomycetales order of Ascomycota have differing substrate preferences enabling more complete biomass utilization.

20. The process of claims 1, wherein fermentation rates are enhanced by pretreating the lignocellulosic biomass.
